(19) United States
(12) Patent Application Publication
(10) Pub. No.: US 2006/0051423 A1
(43) Pub. Date: Mar. 9, 2006

(54) Chitosan-based transport system

(75) Inventors: Katja Heppe, Leipzig (DE); Andreas Heppe, Leipzig (DE); Reinhard Schliebs, Eilenburg (DE)

Correspondence Address: PILLSBURY WINTHROP SHAW PITTMAN LLP, P.O. Box 10500, McLean, VA, 22102, US

(73) Assignee: Katja Heppe - Medical Chitosan, Halle (DE)

(21) Appl. No.: 11/202,323

(22) Filed: Aug. 12, 2005

(30) Foreign Application Priority Data
Aug. 13, 2004 (DE) 10 2004 040 243.4

(51) Int. Cl. A61K 9/14; C08B 37/08

(52) U.S. Cl. 424/488; 536/20; 514/55

(57) ABSTRACT

The invention relates to a chitosan-based transport system for overcoming the blood-brain barrier. This transport system can convey active agents or markers into the brain. The transport system contains at least one substance selected from the group consisting of chitin, chitosan, chitosan oligosaccharides, glucosamine, and derivatives thereof, and optionally one or more active agents and/or one or more markers and/or one or more ligands.

### BACKGROUND

This invention claims priority under 35 USC § 119(a) to German Patent Application No. 10 2004 040 243.4 filed Aug. 13, 2004, herein incorporated by reference in its entirety.

It is one of the great goals of pharmaceutical research to make the various barriers in the body selectively passable for specific substances. These include the intestine-blood barrier, the skin-blood barrier, the nasal mucosa-blood barrier, and the blood-brain barrier (BBB). Problem to be solved.

The blood-brain barrier (BBB) is one of the most problematic barriers as it has highly selective transport systems and as these cells are very tightly joined. The blood-brain barrier is formed by the endothelium of the capillary vessels. These endothelial cells adhere by tight junctions and prevent entry of polar substances exceeding a specific molecular weight into the brain. However, some nutrients (such as D-glucose) and hormones overcome the blood-brain barrier using selective transport systems. Tight junctions (Latin: zonulae occludentes) are strip-shaped junctions of cell membranes that appear to be so tight under the electron microscope as if the membranes were fused. However, actual contact only occurs among the proteins embedded in the outer layer of the participating cell membranes. The protein involved is occludin, a transmembrane protein. The tight junctions occur over extremely short sections of a few nanometers that belong—as becomes visible in freeze breaks only—to a network of globular occludin molecules arranged in a chainlike order which “weld” the epithelial cells to each other.

A particular problem is the transport of hydrophilic substances through the BBB. Pharmaceutical researchers therefore are looking for ways to encapsule such hydrophilic substances in lipophilic particles or bind them to particles with substances that permit receptor-mediated transport across the BBB.

In recent years, they increasingly worked on transport systems consisting of nanoparticles. Nanoparticles mostly consist of polymers and are about 10 to 1000 nm in size. See Kreuter, Journal of Anatomy 1996, 189, pp. 503-505. Some researchers managed to produce efficient nanoparticles that ensure rapid transport of drug-charged particles across the BBB. Nanoparticles from polybutyl cyanoacrylate are able to transport drugs by encapsulating or binding them to the surface of the nanoparticles. See Schroeder et al., Journal of Pharmaceutical Science 1998, 87, 11, pp. 1305-1307 Schroeder et al., Progress in Neuro-Psychopharmacology and Biological Psychiatry 1999, 23, pp. 941-949; Alyautdin et al., Pharmaceutical Research 1997, 14, 3, pp. 325-328; and Ramge et al., European Journal of Neuroscience 2000, 12, pp. 1931-1940. However, these nanoparticles cannot be transported across the BBB directly, only by coating them with polysorbate 80. See Kreuter, Advanced Drug Delivery Reviews 2001, 47, pp. 65-81; and Kreuter, Current Medicinal Chemistry-Central Nervous System Agents 2002, 2, pp. 241-249. Nanoparticles consisting of polycyanoacrylate that were coated with polyethylene glycol could only overcome the BBB if, due to an infection of the brain, the BBB is defective and has become less permeable. See Calvo et al., European Journal of Neuroscience 2002, 15, pp. 1317-1326]. Wang et al. (Molecular Therapy 2001, 3, 5, pp. 658-664) found a cationic polymer (polyethylenimine) with which you can bypass the BBB and use an intramuscular injection in the tongue to introduce drugs into the brain using retrograde axonal transport. Rousselle et al. (Molecular Pharmacology 2000, 57, pp. 679-686) transported doxrubicin across the BBB using a peptide vector. The drug to be transported is covalently bound to D-penetrantin, a peptide, and synB1, which facilitates transport across the BBB without causing ejection by the P-glycoprotein. Other ways include transporting nanoparticles via the transferrin receptor by binding them to ligands. See Li et al., Trends in Pharmacological Sciences 2002, 23, 5, pp. 206-209. This system however has the setback that you can charge the particles with a small quantity of the substance to be transported only.

The previous results of nanoparticle research have shown that neither complicated manufacturing processes nor damage to the BBB is required to transport hydrophilic substances into the brain or that the coating substances are insufficiently decomposed and/or decomposed into harmful monomers.

Many prior art systems are based on coating materials that are composed of one or several cationic and/or anionic layers. Previous approaches were based on the assumption that the transport systems must be physically and chemically stable to protect their content (active agents) and take them to their destination. This is why most systems have very good mechanical properties and do not or do not readily dissolve in the bloodstream.

The use of monosaccharides for overcoming the blood-brain barrier was studied to some extent. See U.S. Pat. No. 6,294,520, which describes oral administration of monosaccharides and amino acids, among other purposes, for supporting the treatment of hair loss.

If an active ingredient has entered the bloodstream it can be metabolized by the liver, discharged by the kidney, or passed to the intestine by the gall bladder. This is why a high dose is often needed to get the required effective quantity to the affected tissues.

Chitosan has been known for some years now as a drug delivery system. Chitosan has some interesting properties and is studied in many areas of medicine and pharmaceutics. It is known that nanoparticles with chitosan coats or nanocapsules can transport pharmaceuticals into the body or overcome the skin-blood or intestine-blood barrier. These barriers are overcome relatively easily. However the blood-brain barrier (BBB) is one of the most problematic barriers to overcome as it has highly selective transport systems and as the cells are very tightly joined.

It is therefore the problem of the invention to provide a chitosan-based transport system for overcoming the blood-brain barrier. This transport system is to convey active agents or markers into the brain. Summarize independent claims.

### BRIEF SUMMARY OF THE INVENTION

This invention relates to a transport system containing (a) at least one substance selected from the group consisting of chitin, chitosan, chitosan oligosaccharides, glucosamine, and derivatives thereof; and (b) optionally one or more active agents and/or one or more markers and/or one or more ligands.

### BRIEF DESCRIPTION OF THE DRAWINGS

FIG. 1: Various embodiments of the transport system are shown.

FIG. 2: A fluorescence microscopic picture of a section through the brain of a mouse in the hippocampus region (the bar represents 100 μm) is shown.

FIG. 3: A close-up shot of a neuronal cell comprising a high content of particles of the transport system (black coloration) (the bar at the bottom right in the figure represents 100 μm.) is shown.

FIG. 4: A fluorescence microscopic picture of a neuronal cell from the brain of a mouse is shown. The stained cells are shown in dark gray. The transport system is shown in black (the bar represents 50 μm).

FIG. 5: A fluorescence microscopic picture of a tissue slice with neuronal cells is shown. The cells are stained gray, and the transport system appears in the form of black dots (the bar represents 50 μm).

FIG. 6: A fluorescence microscopic picture of a tissue slice with neuronal cells is shown. The cells are stained gray, and the transport system appears in the form of black dots (the bar represents 50 μm).

FIG. 7: A fluorescence microscopic picture of a tissue slice with β-amyloid plaque (2) is shown where the transport system (1) has accumulated (the bar represents 50 μm).

FIG. 8: A section of an electron microscopic image of a neuronal cell is shown. The transport system has accumulated at the nuclear-investing membrane and is indicated by a circle.

### DETAILED DESCRIPTION OF THE INVENTION

Basic units of the transport system are building blocks of chitin, chitosan, chitosan oligosaccharides, and glucosamine or their derivatives. The term “chitosan oligosaccharide” includes both carbohydrates that contain up to 10 monosaccharides and longer-chain polysaccharides. Chitosan may be obtained from natural chitin by deacetylating the amide bond, the degree of deacetylation (DDA) being controllable. Chain length and molecular weight of chitosan oligosaccharides can also be accurately set during preparation. WO 03/029297 A2 describes a suitable method. Chitin, chitosan, or chitosan oligosaccharides can have different properties depending on chain length and degree of deacetylation. Both parameters, chain length and degree of deacetylation, can be set during preparation using procedures known by those of skill in the art.

Chitosan oligosaccharides, chitosan, and chitin with molecular weights from 179 Da (glucosamine) to 400 kDa are preferably used for the transport system of the invention (Da=dalton). It is more preferred that the chitosan oligosaccharides, chitosans, and chitins have molecular weights from 179 Da to 100 kDa. Particularly preferred are chitosan oligosaccharides, chitosans, and chitins with molecular weights from 179 Da to 1.8 kDa and chain lengths of 1 to 10 N-acetyl glucosamine or glucosamine rings.

Most preferred are chitosan oligosaccharides, chitosans, and chitins with molecular weights from 800 Da to 1.8 kDa and chain lengths of 5 to 10 N-acetyl glucosamine or glucosamine rings.

Chitin, the chitosans, and chitosan oligosaccarides have degrees of deacetylation from 0 to 100%.

The preferred degree of deacetylation (DDA) is in the range from 30 to 100%. Particularly preferred is a degree of deacetylation of 70 to 100%.

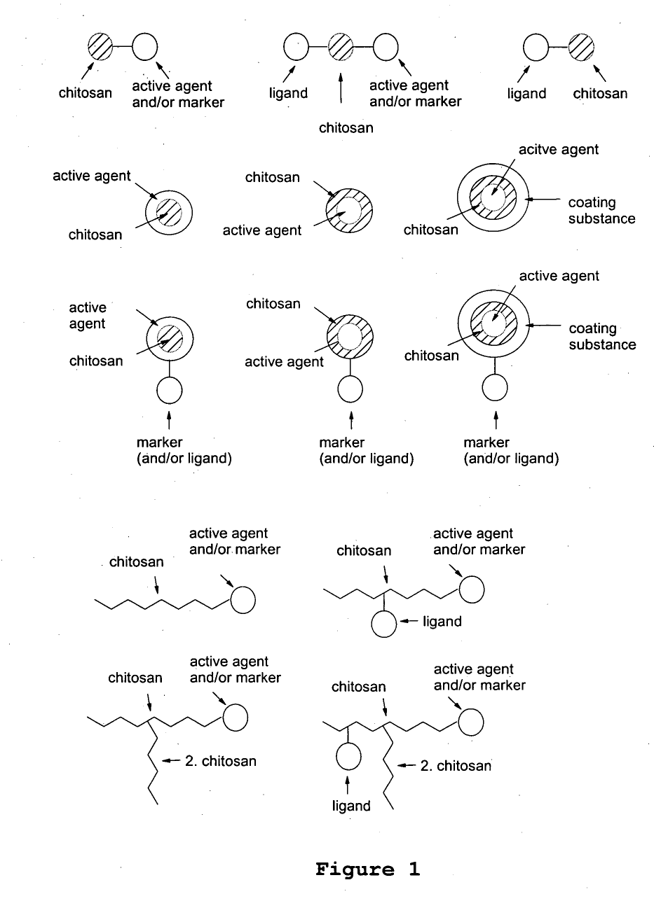

Active agents and/or markers and/or ligands can be bound in various ways to the basic units using methods known by those of skill in the art. A preferred way is binding via the NH₂ group of the glucosamine rings. FIG. 1 shows diagrams of various options. Chitin, chitosan, chitosan oligosaccharide, glucosamine or their derivative are generally referred to by the term “chitosan” in FIG. 1. These ligands are used to dock to the receptors.

The transport system according to the invention preferably is designed in such a way that the chitin, chitosan, chitosan oligosaccharide, glucosamine or their derivative is bound to one or more active agents and/or one or more markers. One or more ligands may be bound instead of active agents and/or markers, or, optionally, in addition to them.

In another preferred embodiment, the transport system is designed so that the chitin, chitosan, chitosan oligosaccharide, glucosamine or their derivative is coated by one or more active agents. Likewise, one or more active agents can be coated by the chitin, chitosan, chitosan oligosaccharide, glucosamine or their derivative. This substance may preferably be coated by another coating substance. Preferred coating substances are starch and/or alginate.

It is preferred that one or more markers and/or one or more ligands are bound to the coat of chitin, chitosan, chitosan oligosaccharide, glucosamine or their derivative or to the coat of active agent.

In this context, if another coating substance is present, it is preferred that one or more markers and/or one or more ligands are bound to the outer coat.

In another embodiment, the chitin, chitosan, chitosan oligosaccharide, glucosamine or their derivative is present as a chain and is bound to one or more active agents and/or one or more markers.

It is preferred in this embodiment of the transport system that chain-like chitin, chitosan, chitosan oligosaccharide, glucosamine or their derivative are bound to the chitin, chitosan, chitosan oligosaccharide, glucosamine or their derivative.

It is particularly preferred in the two latter embodiments (chain) that one or more ligands are bound to the chitin, chitosan, chitosan oligosaccharide, glucosamine or their derivative.

The bound ligands can connect to receptors on membranes. These substances preferably are substances from the group of transferrin, insulin, insulin-like growth factors, and polysorbate-80.

The transport system preferably contains substances as active ingredient that develop an effect in the brain.

The transport system is preferably solid, liquid, or semisolid.

It can be applied by oral, dermal, or parenteral administration (preferably by intravenous injection).

The transport system overcomes body barriers (dermal, oral, etc.) and enters the vascular system. The vascular system transports the particles, capsules, or molecules that partially re-arrange into specific cells or extracellular structures. Surprisingly, it has been found that this also occurs across the blood-brain barrier.

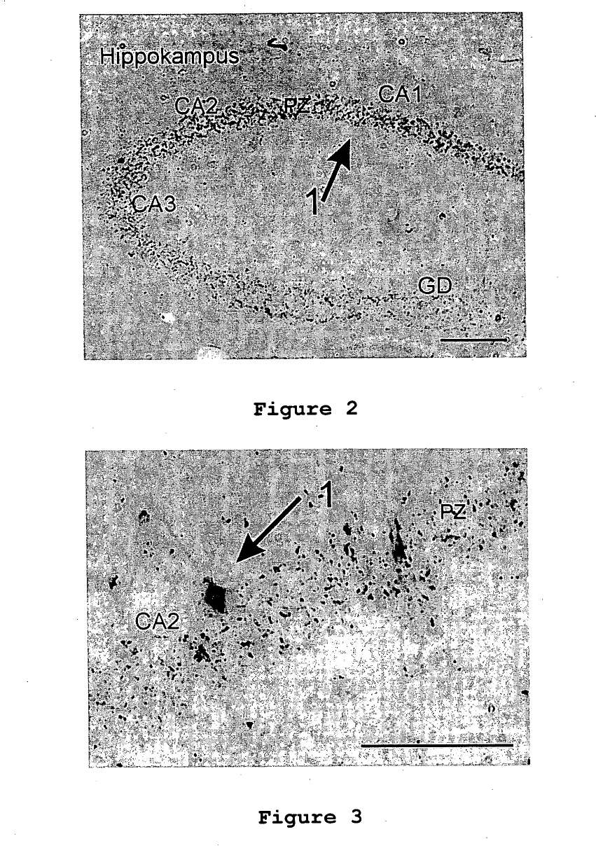

Absorption in the brain could be proven by studies in mice that had the transport system according to the invention containing a fluorescent marker injected intravenously. FIG. 2 shows the fluorescence microscopic picture of a section through the brain from the area of the hippocampus. The hippocampus is the region in the brain that is responsible for short-term memory, forming associations, and recognition of situations and objects. Considerable changes of this region of the brain occur with diseases such as Alzheimer's disease.

A clear accumulation of fluorescent particles can be seen in the pyramidal cell layers (Pz) of hippocampus subregions CA1, CA2, and CA3. (The bar at the bottom right in the figure represents 100 μm.)

FIG. 3 shows a close-up shot of a neuronal cell comprising a high content of particles of the transport system according to the invention (black coloration) (The bar at the bottom right in the figure represents 100 μm.).

It was found in studies that the composition can be reconfigured in the blood if, for example, long chitin or chitosan components disintegrate into short molecular blocks. It was found surprisingly that the blood itself and primarily the erythrocytes in it can assume a filtering or sorting function causing the chitin or chitosan molecule chains to disintegrate into molecular blocks with preferably 4 to 10 chitin or chitosan rings (N-acetyl glucosamine or glucosamine rings). These form active agent transport mixtures that can be transported independently and are preferably transported by erythrocytes. Chitin or chitosan molecules having the same structures and molecular size as glucose or glucosamine transported by erythrocytes preferably bind to erythrocytes.

If such a rearrangement occurs in the blood, absorption preferably takes place by glucose transport points at the blood-brain barrier or blood-organ barrier. Depending on the organ and configuration, transport can also be achieved via tight junctions or endocytotic or receptor-mediated processes.

It was found that the transport systems have great affinity for specific cells. These are cells with a high metabolic activity (energy-consuming) cells that are characterized by considerable glucose consumption. In the brain, besides in microgliocytes, particles preferably accumulate in neuronal cells, more preferably in pyramidal neurons.

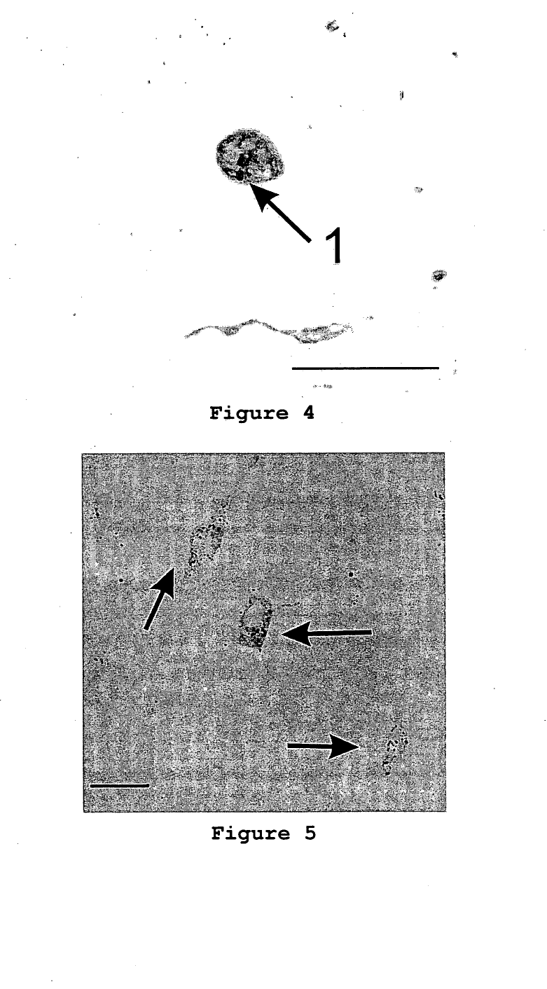

Absorption in the cells can be proven by studies in mice that had the transport system containing a fluorescent marker injected intravenously. FIG. 4 shows the fluorescence microscopic picture of a neuronal cell from the brain of a mouse. The cells stained with cell-specific and fluorescent markers (parvalbumin positive) appear dark gray. The transport system is shown in black (the bar represents 50 μm).

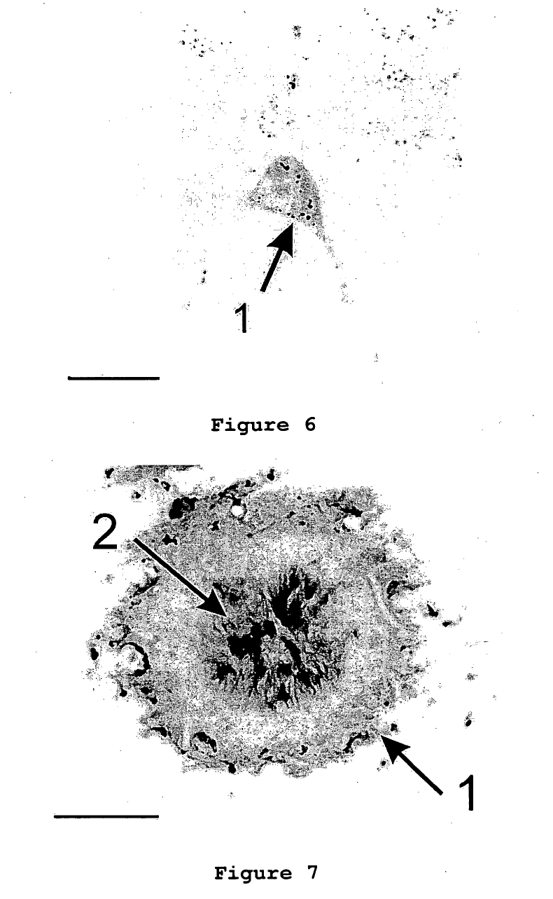

FIGS. 5 and 6 also show fluorescence microscopic pictures of tissue slices with neuronal cells. The cells are stained gray, the transport system appears in the form of black dots (the bar represents 50 μm). It is clearly visible that the transport system (black) is located within the gray area (cell). The picture demonstrates that the transport system is absorbed in the cells.

In addition to this accumulation in and on cells, accumulation at extracellular structures, preferably at structures rich in protein such as β-amyloid plaques in the Alzheimer pathology. FIG. 7 shows a fluorescence microscopic picture of a tissue slice with β-amyloid plaque (2) where the transport system (1) has accumulated (the bar represents 50 μm).

It can also be absorbed in inflammatory brain regions or in tumor tissue. Absorption is similar in other body tissues. Cells with a high energy metabolism (such as inflammations, tumors) are addressed primarily again.

The mechanism of action of preferred absorption via the glucose transporter in addition to endocytotic and receptor-mediated processes also explains the special effect on active, inflammatory, and tumor cells. These cells have a particularly good growth-related energy demand.

Depending on the modification of the composition, the transport system and active agent are separated in the cell or passed on to other areas of the cell such as lipide-like structures for absorption in or accumulation at organellas such as mitochondria or the nucleus. If the composition or its chitin, chitosan, chitosan oligosaccharide, or glucosamine portion is absorbed in the nucleus, it accumulates at DNA structures.

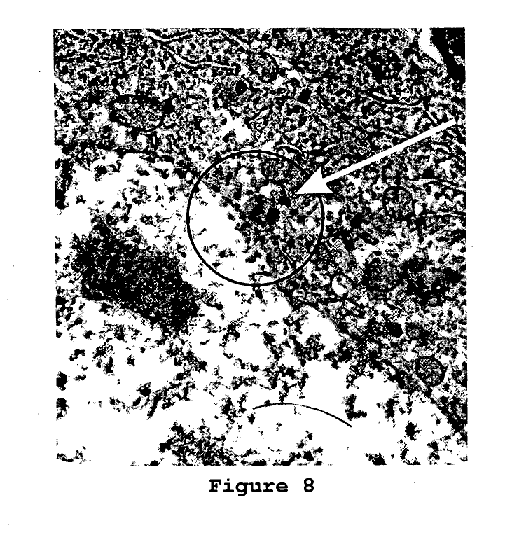

Accumulation sites in the cell can be proven by studies in mice that had the transport system injected intravenously. FIG. 8 shows a section of electron microscopic pictures of neuronal cell. The transport system has accumulated at specific cell structures, in this case, the nuclear-investing membrane, and is indicated by a circle. The fluorescence microscopic picture in FIG. 4 also clearly shows a concentration at specific sites in the cell.

Surprisingly, concentration (clustering) of various individual compositions may occur at extracellular structures.

If chitin, chitosan, chitosan oligosaccharide, or glucosamine (basic elements) and active agent are separated or if only the basic element is introduced into the body, it can cause the following effects:

Accumulation at or depositing in the membranes of cells and/or organellas and the resulting influence on signal cascades.

Change in absorption or discharge of substances of any kind such as growth factors, messenger substances, minerals, electrolytes, and others in or from the cell.

These effects can also occur without separation of the basic element from the active agent.

In addition to causing an effect in the cells, specific structures that are marked by the basic elements can be identified outside the cell and used for diagnostic purposes. After the diagnosis or unfolding of the effect of the bound substances the transport system decomposes so that the bound substance remains in the cell or is transported as an unbound particle through the vascular system, decomposed, or discharged.

Chitin, chitosan, chitosan oligosaccharide, or glucosamine are decomposed without residue in the cell or in the body.

Thus the transport system can, in a cell-specific manner, dock to, or penetrate into cells that have these features, even outside the brain.

Controlled accumulation of chitin, chitosan, chitosan oligosaccharide, or glucosamine and active agent particles makes it possible to introduce diagnostic or therapeutic agents and transport them to the focus of the disease or the action site. As the transport system accumulates in the metabolically active cells whose metabolism is increased as compared to other cells low doses of diagnostic or therapeutic agents can be administered as these concentrate in the diseased tissues of the body.

In this respect, the transport system can be used to produce an agent for diagnosing brain-specific diseases, such as the diagnosis of tumors and Alzheimer's disease. In addition, the transport system described can be used as a diagnostic or therapeutic agent with malignant brain tumors. For example, highly effective antitumor agents such as tamoxifen can be delivered to the site where the effect should develop.

If the transport system is linked to a radioactive substance, it can be used to diagnose foci of disease (inflammations) or tumors in vivo even if they are present at a low concentration (metastases, tumors in their early stage).

In Alzheimer pathology, a β-amyloid-affine radioactive substance may be bound to the transport system for controlled identification of plaque foci and concentration of diagnostics there. If the marker does not concentrate, it is assumed that there is no pathologic change.

To diagnose beta-amyloid deposits using positron emission tomography (PET) the chitosan transport system may be labeled with C11 by methylating the chitosan. Specific activities of more than 2000 Ci/mmol were targeted.

The transport system can be used for treating brain-specific diseases, such as the treatment of tumors and Alzheimer's disease.

Suitable active agents for treatment are those that can be bound to the transport system and that have the ability to concentrate at the diseased sites and penetrate into the cells. This allows for a relatively low dose in relation to the body, which reduces the side effects of the drugs.

Particularly preferred active agents include acetylcholine precursors, in particular, choline and lecithin, stimulants for acetylcholine release such as linopirdine, acetylcholine sterase inhibitors, in particular, tacrine, donepezile, rivastigmine, metrifonate, and galantamine, muscarine receptor agonists, in particular, xanomeline, milameline, AF102B, Lu25-109, SB202026, and talsaclidine, beta-sheet breakers, neutral endopeptidases such as neprilysine, painkillers, inflammation inhibitors such as propentofylline, ibuprofen, and indomethacin, antioxidants, neuroprotective agents, NMDA antagonists, and antirheumatics.

The nerve growth factor (NGF) is a particularly preferred active agent.

Preferred antioxidants include vitamins E and C; deprenyl (selegiline; MAO-B inhibitor); and gingko biloba.

Preferred neuroprotective agents include Q10, nicotin, cerebrolysin, piracetam, phosphatidyl serine, and acetyl-L-camitine. A particularly preferred NMDA antagonist is memantine.

Chitin or chitosan and chitosan oligosaccharide are decomposed without residue by the organism due to their glucose-like structure. Surprisingly, chirosan resorbed from the urine in the kidney and returned to the body.

As the particles are decomposed without residue, no further load on the organism by harmful monomers occurs, and the monomer that is formed is glucosamine.

It can be expected that the transport system of the invention, due to its capability to be conveyed via glucose transporters and/or the openings of tight junctions, will be able to overcome the blood-blood barrier between mother and fetus. This capability can be utilized at the prenatal stage for diagnostic and therapeutic purposes.

The invention is explained in greater detail with reference to examples, which should not be construed as limiting the claimed invention in any manner.

### EXAMPLES

Example 1—Intravenous administration of a mixture of chain-like chitosan with a molecular weight from 1.8 kDa to 300 kDa and a degree of deacetylation from 80 to 100% to which a peptide or polypeptide of maritime origin was bound for treating tumor diseases in the brain. Administration of 45 mg of active agent per day over a period of 90 days; the transport agent/active agent mixture is absorbed in normal saline and applied.

Example 2—Preparation of chitosan oligomer in pure form and with a low degree of deacetylation (DDA) <80% and a molecular weight from 800 to 1600 Dalton for treating inflammatory diseases in the bloodstream (phlebitis), administration of ≦0.2 mg/100 kg body weight.

Example 3—Preparation of chitosan oligomers with a high DDA >80% molecular weight 500 to 2500 Da and adding 0.2 parts of ibuprofen or indometacin and intravenous administration of ≦0.3 mg/100 kg body weight in 2 ml NaCl solution over 14 days to inhibit inflammations induced by local Alzheimer plaque

Example 4—Preparation of chitosan oligomers with a high DDA and mixing with glucosamine solutions and gingko biloba extract at a ratio of 5:2:1 for oral mucosa penetration (gel film on palatum or lower lip area).

Example 5—Coupling of memantine to chitosan with a degree of deacetylation of 87% and a molecular weight of 1.8 kDa. The transport system is stabilized by another coating with chitosan (DDA 90%) with a molecular weight of 150 kDa. The preparation is administered orally once a day at a dose of 5 mg of active ingredient in the first week that is increased at weekly increments of 2.5 mg to the maximum dose of 15 mg/day.

Example 6—Coupling of donepezile, rivastignine, or galantamine to chitosan transport system (DDA 85%,) so that the administered dose is 2 to 5 mg of active ingredient per day; chronic application over several weeks (40 weeks).

What is claimed is:

1. A transport system for overcoming the blood-brain barrier, comprising:
at least one substance selected from the group consisting of chitin, chitosan, chitosan oligosaccharides, glucosamine, and derivatives thereof; and
optionally, one or more active agents and/or one or several markers and/or one or more ligands.

2. The transport system according to claim 1, wherein the chitosan oligosaccharides, chitosans, and chitins have molecular weights ranging from 179 Da to 400 kDa.

3. The transport system according to claim 2, wherein the chitosan oligosaccharides, chitosans, and chitins have molecular weights ranging from 179 Da to 100 kDa.

4. The transport system according to claim 3, wherein the chitosan oligosaccharides, chitosans, and chitins have molecular weights ranging from 179 Da to 1.8 kDa and chain lengths of 1 to 10 N-acetyl glucosamine or glucosamine rings.

5. The transport system according to claim 4, wherein the chitosan oligosaccharides, chitosans, and chitins have molecular weights ranging from 800 Da to 1.8 kDa and chain lengths of 5 to 10 N-acetyl glucosamine or glucosamine rings.

6. The transport system according to claim 5, wherein the chitins, chitosans and chitosan oligosaccharides have a degree of deacetylation ranging from 0 to 100%.

7. The transport system according to claim 6, wherein the chitins, chitosans and chitosan oligosaccharides have a degree of deacetylation ranging from 30 to 100%.

8. The transport system according to claim 7, wherein the chitins, chitosans and chitosan oligosaccharides have a degree of deacetylation ranging from 70 to 100%.

9. The transport system according to claim 1, wherein the substance is bound to one or more active agents and/or one or more markers.

10. The transport system according to claim 1, wherein the substance is bound to one or more ligands.

11. The transport system according to claim 1, wherein the substance is coated by one or more active agents.

12. The transport system according to claim 1, wherein one or more active agents are coated by the substance.

13. The transport system according to claim 12, wherein the transport system is coated by a second coating substance.

14. The transport system according to claim 13, wherein the second coating substance is a starch, an alginate, or mixtures thereof.

15. The transport system according to claim 11, wherein one or more markers and/or one or more ligands are bound to the coating.

16. The transport system according to claim 12, wherein one or more markers and/or one or more ligands are bound to the coating.

17. The transport system according to claim 13, wherein one or more markers and/or one or more ligands are bound to the second coating.

18. The transport system according to claim 1, wherein the substance is present in form of a chain and bound to one or more active agents and/or one or more markers.

19. The transport system according to claim 18, wherein chain-like chitin, chitosan, chitosan oligosaccharide, glucosamine or derivatives thereof are bound to the substance.

20. The transport system according to claim 18, wherein one or more ligands are bound to the substance.

21. The transport system according to claim 19, wherein one or more ligands are bound to the substance.

22. The transport system according to claim 1, wherein the active agents are substances that develop an effect in the brain.

23. The transport system according to claim 22, wherein the active agents are selected from the group consisting of acetylcholine precursors, stimulants for acetylcholine release, acetylcholine esterase inhibitors, muscarine receptor agonists, beta-sheet breakers, neutral endopeptidases, painkillers, inflammation inhibitors, antioxidants, neuroprotective agents, NMDA antagonists, antirheumatics, nerve growth factors, and combinations thereof.

24. The transport system according to claim 1, wherein the ligands are selected from the group consisting of transferrin, insulin, insulin-like growth factors, polysorbate-80, and combinations thereof.

25. A method of using the transport system according to claim 1 to treat brain-specific diseases.

26. The method according to claim 25, wherein the brain-specific diseases are tumors.

27. The method according to claim 25, wherein the brain-specific disease is Alzheimer's disease.

28. A method of using the transport system according to claim 1 to prepare a diagnostic agent for brain-specific diseases.

29. The method according to claim 28, wherein the method is used for tumor diagnosis.

30. The method according to claim 28, wherein the method is used for diagnosing Alzheimer's disease.
